(19) United States
(12) Patent Application Publication
(10) Pub. No.: US 2026/0221267 A1
(43) Pub. Date: Jul. 30, 2026

(54) PLATFORM FOR INTEGRATING AND PUBLISHING HETEROGENEOUS BIOMEDICAL DATA WITH SEMANTIC ANNOTATION

(71) Applicant: Shuhan He, Skokie, IL (US)

(72) Inventor: Shuhan He, Skokie, IL (US)

(21) Appl. No.: 19/461,107

(22) Filed: Jan. 27, 2026

Related U.S. Application Data
(60) Provisional application No. 63/751,632, filed on Jan. 30, 2025.

(51) Int. Cl. G16H 30/40; G16H 30/20

(52) U.S. Cl. CPC G16H 30/40; G16H 30/20

(57) ABSTRACT

Embodiments of the present invention provide a platform for integrating and publishing heterogeneous biomedical data, such as genomic, proteomic, clinical, and imaging data, by employing semantic annotation techniques. The platform enables the efficient mapping, harmonization, and linkage of data from diverse biomedical sources through standardized ontologies and semantic metadata. The platform facilitates the publication of this integrated data in a machine-readable, standardized format, enhancing the discoverability, interoperability, and analysis of biomedical data for research and clinical decision-making. Additionally, the platform includes a semantic procurement subsystem that transforms Methods sections into machine-actionable resource specifications, generates bills of materials and services, and enables direct procurement actions with referral tracking and attribution.

### TECHNICAL FIELD

The present invention relates generally to a publishing infrastructure. More specifically, the present invention relates to a platform for integrating and publishing heterogeneous biomedical data with semantic annotation.

### BACKGROUND ART

In the realm of digital publishing, a significant challenge has emerged with the evolution of publication formats and data management. Traditional legacy formats, such as PDF, printed documents, and proprietary file formats, present significant obstacles when it comes to the efficient representation, accessibility, and interaction of structured data. This challenge is particularly acute in biomedical research, where publications contain complex data types, including genomic sequences, medical images, and electrophysiological recordings that lose critical functionality when converted to static formats.

A key problem faced in these legacy formats is the "flattening" of data. Flattening refers to the conversion of structured data, such as tables, charts, genomic variants, imaging metadata, and other complex elements, into static, unstructured formats that strip away the underlying relationships and hierarchical organization. As a result, users can no longer interact with the data in its original form, such as querying, searching, or extracting specific values. The rich contextual information that was present at the point of data acquisition becomes inaccessible once published.

This flattening process often occurs as a consequence of presenting data in a visually formatted and consistent manner that is easy to read on a variety of devices. Unfortunately, this simplification leads to the loss of important metadata, cross-references, and interactive elements. The integrity of the data may be compromised, and the user's ability to manipulate or reuse the data becomes severely limited. What was once a dynamic, queryable dataset becomes a static representation that requires manual re-extraction to be useful.

The data flattening problem is particularly acute in biomedical research, where data plays a critical role in scientific discovery and clinical decision-making. The inability to retain, represent, and process data in a structured way within legacy formats creates inefficiencies, inaccuracies, and difficulties in data analysis. Researchers and clinicians who wish to build upon published findings must undertake laborious manual processes to recover the structured data that was lost during publication.

Research articles and reports frequently contain tables and figures that, once flattened, lose important details such as references to specific data points, interactive charts, or formulas. Genomic data, imaging data, electrophysiological recordings, and other domain-specific data types are particularly affected. For example, genomic data that was once rich with context, such as clickable variant information, links to functional annotations, and integration with related datasets, becomes a static, unstructured body of text when published in traditional formats. These documents are not clickable, lack contextual depth, and fail to provide direct access to underlying datasets or additional metadata.

This outdated publication model significantly impedes the ability of clinicians and researchers to leverage new biomedical insights. Without clickable links, standardized metadata, or machine-readable formats, any attempt to use published information in subsequent research or clinical applications requires laborious re-extraction, re-annotation, and reformatting of data that was already standardized and annotated at earlier stages. The burden of manually converting information back into usable datasets falls on individual researchers and clinicians, leading to redundancy, errors, and massive inefficiencies across the scientific community.

Furthermore, with the NIH Data Management and Sharing (DMS) Policy now demanding accessible, interoperable, and reusable data outputs, it is no longer sustainable for publications to be mere textual endpoints. Funding agencies and regulatory bodies increasingly require that published research data remain findable, accessible, interoperable, and reusable (FAIR). Legacy publication formats fundamentally conflict with these requirements.

Beyond the challenges of data flattening, current biomedical publications suffer from a fundamental limitation in operational reproducibility: the Methods sections of research articles remain unstructured prose that is not machine-actionable. While Methods sections describe the procedures, reagents, instruments, software, and services used in research, this information is presented as free-form text that cannot be directly parsed, validated, or acted upon by computational systems. This limitation creates a second major barrier to effective scientific communication and reproducibility.

Resource identification within Methods sections is inherently ambiguous. Authors may reference reagents, antibodies, cell lines, instruments, or software without providing complete identifying information, such as vendor names, catalog numbers, lot numbers, software versions, instrument settings, or configuration parameters. Even when such details are included, they appear in inconsistent formats and locations within the text, making systematic extraction unreliable. This ambiguity extends to datasets, where accession numbers, version identifiers, and access conditions may be incompletely specified or difficult to locate within the prose.

As a consequence, reproduction of published research is significantly impaired. Researchers attempting to replicate a study must manually parse the Methods section, identify each resource mentioned, and independently source those materials through separate procurement channels. This process is error-prone and inconsistent, as researchers may inadvertently substitute different reagent lots, software versions, or instrument configurations that affect experimental outcomes. The lack of standardized resource identification contributes directly to the well-documented reproducibility crisis in biomedical research.

Furthermore, there is no standardized mechanism in current publication systems for creating persistent "resource objects" that link experimental materials and tools to purchase, licensing, or access workflows. Each resource mentioned in a Methods section exists only as text, without structured connections to vendor catalogs, institutional procurement systems, software repositories, or data access portals. Researchers must manually navigate multiple external systems to acquire the resources needed for replication, with no guarantee that they are obtaining the exact materials used in the original study.

This fragmented approach produces significant inefficiencies and contributes to misreplication of research findings. Without machine-readable resource specifications, it is impossible to automatically generate a "bill of materials" or "bill of services" that comprehensively lists all resources required to reproduce a study. Institutions cannot leverage their existing vendor contracts or preferred supplier relationships when researchers attempt to source materials from publications. The disconnect between publication content and procurement infrastructure represents a substantial barrier to efficient, accurate research replication.

Therefore, there is a critical need for a solution that can preserve the structure and relationships of data in publication formats, enable interaction and querying of data without requiring manual re-entry or reformatting, facilitate integration of data into modern workflows and analytical tools without losing contextual information, and seamlessly integrate structured non-text biomedical data (imaging, genomic variant, electrophysiological data) in forms such as VCF, DICOM, NWB, and HPC log files into the manuscript itself, ensuring that the final published article remains machine-readable and interactive with embedded metadata. Furthermore, there exists a need for a platform that addresses both the data flattening problem and the Methods section reproducibility problem in an integrated manner.

### OBJECTS OF THE INVENTION

Some of the objects of the invention are as follows:

An object of the present invention is to create a comprehensive, NIH-compliant metadata schema aligning manuscript sections, figures, tables, and methods with UMLS, VSAC, RxNorm, and SNOMED CT codes for seamless repository integration.

Another object of the present invention is to provide a platform that recognizes and ingests specialized biomedical file types (VCF, DICOM, NWB, etc.) in tandem with textual manuscripts.

Another object of the present invention is to implement AI models in the publishing platform to assign UMLS-based codes and relevant VSAC, RxNorm, and SNOMED CT mappings to biomedical terms in both new and legacy publications.

Another object of the present invention is to provide a publishing platform that publishes the raw context-rich data within the manuscript in an interactive, machine-readable format, not merely as supplemental data. This includes VEP (Variant Effect Predictor Data), electrophysiology data (NWB), and others.

Another object of the present invention is to provide a publishing platform that integrates the Variant Effect Predictor (VEP) into the platform to automatically annotate genomic variants from TCGA VCF files.

Another object of the present invention is to provide a publishing platform that enhances annotated data with standardized medical terminologies (UMLS, VSAC, RxNorm, SNOMED CT).

Another object of the present invention is to provide a platform that automatically formats, stores, and indexes genomic data in a FAIR-compliant and NIH DMS (Data Management Standards) JSON outputs upon manuscript submission, promoting data discoverability and reusability in clinical settings.

Another object of the present invention is to provide a secure, compliant infrastructure capable of managing diverse genomic datasets, ensuring efficient data discovery, access, and reuse.

Another object of the present invention is to provide an interoperability framework allowing seamless data exchange between the platform and external genomic databases, facilitating clinical applications and ethical data reuse.

Another object of the present invention is to provide a computer-vision-assisted data capture engine associated with the semantic annotation platform that automatically produces verified metadata from images, videos, or other sensor input at the point of capture, thereby ensuring data authenticity and preventing fraud or manipulation.

Another object of the present invention is to implement a secure chain of custody framework using cryptographic hashing, AI-driven anomaly detection, and version tracking so that each captured dataset (eg, Western blot image, microscopy frame, or behavioral video) can be definitely linked to its original, unaltered source.

Another object of the present invention is to integrate the computer-vision-assisted data capture engine with the semantic annotation platform, wherein the entire workflow (capture, analysis, authentication, and final dissemination) deters fraudulent manipulations by flagging suspicious edits or inconsistencies at the time of submission and peer review.

Another object of the present invention is to publish this verified metadata, along with the associated raw files, in a machine-readable, standards-compliant format, enabling peer reviewers and readers to confirm data integrity from initial capture through final publication.

Another object of the present invention is to ensure that each dataset’s contextual metadata (e.g., antibody details, exposure parameters, time stamps, experimental conditions) travels with the raw data, allowing future researchers to quickly re-check authenticity and reproducibility without needing complicated or manual re-tracing of experimental steps.

Another object of the present invention is to transform Methods sections into semantically tagged workflows that resolve resources, including reagents, instruments, and software, to canonical identifiers and vendor SKUs, enabling machine-readable resource specification within publications.

Another object of the present invention is to generate an interactive "bill of materials" and "bill of services" (BOM/BOS) embedded within the publication that comprehensively lists all resources required to reproduce a study.

Another object of the present invention is to enable direct routing from method elements to procurement endpoints, including purchase, quote request, license acquisition, and data access request workflows for each identified resource.

Another object of the present invention is to track attribution and referral credit for purchases, licenses, or access requests initiated from the publication, recording transaction metadata that links procurement actions back to the publication, author, institution, or platform.

Another object of the present invention is to provide institution-aware purchasing flows that leverage preferred vendor relationships, contract pricing, and institutional approval workflows when researchers source materials from publications.

Another object of the present invention is to generate machine-readable resource objects with persistent identifiers that link experimental materials, software, datasets, and services to their respective procurement, licensing, or access workflows.

### SUMMARY OF THE INVENTION

The invention addresses the need for an integrated platform that can handle diverse biomedical data types, applying semantic annotation to each dataset to provide meaning and structure. By incorporating standard biomedical ontologies and semantic technologies, the platform enables seamless data integration across multiple domains. It provides a unified repository that supports querying, visualization, and sharing of annotated biomedical data in a way that fosters collaboration and accelerates scientific discovery. The platform also includes tools for publishing and sharing data in formats that are compatible with industry standards, ensuring broad accessibility and usability. Additionally, the platform includes a commerce and referral subsystem that uses semantic annotations extracted from Methods sections to create structured resource objects, generate bills of materials and services, and enable direct procurement actions, licensing requests, and data access workflows while preserving compliance, provenance, and journal workflows.

According to a first aspect of the present invention, a semantic annotation platform for research and publication is provided. The platform comprising: a user interface configured to receive at least one textual manuscript file and at least one biomedical data file, wherein the at least one biomedical data file is selected from the group consisting of DICOM imaging data, VCF genomic variant data, NWB electrophysiological data, and non-textual data; a semantic annotation engine configured to extract structured metadata from the at least one biomedical data file by applying at least one standardized clinical or biomedical ontology; wherein the platform incorporates said structured metadata into a semantically enriched article layout that remains machine-readable; and wherein the platform produces an interactive publication output that simultaneously renders textual narrative and references to the structured biomedical data.

In one embodiment of the invention, the interactive publication output is compliant with the NIH Data Management and Sharing Policy.

In one embodiment of the invention, the semantic annotation engine is further configured to embed machine-readable metadata, assign persistent identifiers, and link data to at least one terminology selected from the group consisting of UMLS, VSAC, RxNorm, and SNOMED CT.

In one embodiment of the invention, the semantic annotation engine uses natural language processing and pattern recognition algorithms to classify content into predefined sections and enhance metadata annotation.

In one embodiment of the invention, supplementary materials, including figures, tables, and video recordings, are automatically identified and separated from the main text while maintaining links to relevant sections.

In one embodiment of the invention, metadata for figures, tables, and supplementary materials is enriched with at least one standard selected from the group consisting of NBO, NWB, and UMLS identifiers to support cross-study analyses and interoperability.

In one embodiment of the invention, the semantic annotation engine applies AI-driven tagging to legacy publications to align metadata with current standards.

In one embodiment of the invention, the semantic annotation platform further comprising a guided interface for users to verify and adjust AI-generated tags.

In one embodiment of the invention, the semantic annotation engine comprises a cross-ontology linking mechanism that ensures metadata interoperability by mapping terms across multiple ontologies.

In one embodiment of the invention, metadata and data annotations include provenance and licensing information to ensure ethical reuse and regulatory compliance.

In one embodiment of the invention, the semantic annotation platform further comprising APIs and export functionality for integration with external databases and tools.

According to a second aspect of the invention, a method for automated publication of heterogeneous biomedical data is provided. The method comprising: receiving, via a user interface, at least one textual manuscript file and at least one biomedical data file selected from the group consisting of DICOM imaging data, VCF genomic variant data, NWB electrophysiological data, and non-textual data; extracting structured metadata from the at least one biomedical data file using a semantic annotation engine that applies at least one standardized clinical or biomedical ontology; incorporating the structured metadata into a semantically enriched article layout that remains machine-readable; and producing an interactive publication output that simultaneously renders textual narrative and references to the structured biomedical data.

In one embodiment of the invention, the method further comprises extracting method elements from a Methods section of the textual manuscript file, resolving each method element to at least one resource object comprising a canonical identifier and a commercial identifier, and generating a bill of materials or bill of services listing resources required to reproduce a study.

In one embodiment of the invention, the method further comprises linking each resource object to at least one procurement endpoint selected from the group consisting of purchase, quote request, license acquisition, institutional requisition, data access request, and service scheduling.

In one embodiment of the invention, the method further comprises generating a referral identifier for each procurement endpoint and recording transaction metadata when a procurement action is initiated from the publication.

In one embodiment of the invention, resolving each method element comprises applying natural language processing to identify resource mentions and tagging each resource with ontology identifiers and commercial identifiers.

In one embodiment of the invention, the interactive publication output is compliant with the NIH Data Management and Sharing Policy.

According to a third aspect of the invention, a semantic annotation platform for research publications with integrated procurement functionality is provided. The platform comprising: a semantic annotation engine configured to extract structured metadata from a manuscript file using at least one standardized biomedical ontology; a method element extraction module configured to parse a Methods section of the manuscript into discrete method steps and resource elements; a resource resolution module configured to resolve each resource element to a resource object using the semantic annotation engine, the resource object comprising a canonical identifier from the standardized biomedical ontology and a commercial identifier; a bill of materials generator configured to generate a bill of materials and a bill of services listing resources required to reproduce a study described in the manuscript; a procurement interface module configured to link each resource object to at least one procurement endpoint; and a transaction tracking module configured to record transaction metadata when a procurement action is initiated from the publication.

In one embodiment of the invention, the procurement endpoint is selected from the group consisting of purchase, quote request, license acquisition, institutional requisition, data access request, and service scheduling.

In one embodiment of the invention, the transaction tracking module is further configured to generate referral identifiers embedded within the publication and attribute downstream procurement actions back to the publication, author, institution, or platform.

In the context of the specification, the phrase “module” as used herein refers to any known or later developed hardware, software, firmware, artificial intelligence, fuzzy logic, or combination of hardware and software that is capable of performing the functionality associated with that element. Also, while the disclosure is presented in terms of exemplary embodiments, it should be appreciated that individual aspects of the disclosure can be separately claimed.

In the context of the specification, the phrase “JavaScript Object Notation (JSON)” refers to a format used for storing and exchanging structured data. It is based on JavaScript Object syntax but is language-independent. A JSON object can contain data represented as key-value pairs, nested objects, and arrays, and written as plain text.

In the context of the specification, the term “processor” refers to one or more of a microprocessor, a microcontroller, a general-purpose processor, a Field Programmable Gate Array (FPGA), a Graphics Processing Unit (GPU), a Neural Processing Unit (NPU), a Tensor Processing Unit (TPU), an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non- volatile storage memory such as EPROM, EEPROM, flash memory, or the like.

In the context of the specification, the phrase "resource object" refers to a structured data entity that captures comprehensive information about a material, instrument, software, dataset, or service referenced in a publication, including identifying information, commercial identifiers, and linkages to procurement endpoints.

In the context of the specification, the phrase "method element" refers to a discrete component extracted from a Methods section, which may be either a method step representing a protocol action or a resource element representing an input, tool, or service required to perform that action.

In the context of the specification, the phrase "procurement endpoint" refers to an interface or service that enables acquisition of a resource, including but not limited to purchase transactions, quote requests, license acquisitions, institutional requisitions, data access requests, and service scheduling.

In the context of the specification, the phrase "referral identifier" refers to a unique identifier embedded within a publication or procurement link that enables attribution of downstream procurement actions back to the publication, author, institution, or platform.

In the context of the specification, the phrase "transaction metadata" refers to recorded information about procurement actions initiated from a publication, including the relationship between the publication and the action, without necessarily including payment processing details.

In the context of the specification, the phrase "bill of materials" (BOM) refers to a comprehensive listing of physical resources, reagents, and materials required to reproduce a study as extracted from the publication.

In the context of the specification, the phrase "bill of services" (BOS) refers to a comprehensive listing of services, software licenses, and data access requirements needed to reproduce a study as extracted from the publication.

In the context of the specification, the phrase "purchase workflow" refers to a sequence of actions and interfaces that enable a user to acquire a resource, which may include adding items to a cart, selecting vendors, applying institutional pricing, obtaining approvals, and completing transactions.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a conventional research publication platform.

FIG. 2 illustrates a user interface for uploading a document, in accordance with an embodiment of the present invention.

FIG. 3 is a block diagram showing a process flow for automated publication of heterogeneous biomedical data, in accordance with an embodiment of the present invention.

FIG. 4 is a block diagram showing an architecture of a semantic annotation platform for the automated publication of heterogeneous biomedical data in accordance with an embodiment of the present invention.

FIG. 5 is a flow diagram showing a semantic procurement and referral subsystem for transforming Methods sections into machine-actionable procurement interfaces, in accordance with an embodiment of the present invention.

FIG. 6 is a flow diagram showing the manuscript submission and peer review tracking integrated in the semantic annotation platform, in accordance with an embodiment of the present invention.

FIG. 7 shows a flow diagram illustrating a method for ingesting Variant Call Format (VCF) files and integrating metadata to support enhanced biomedical data management and analysis, in accordance with an embodiment of the present invention.

FIG. 8 shows a flow diagram illustrating a method for ingesting DICOM (Digital Imaging and Communications in Medicine) files and integrating relevant metadata to support enhanced biomedical data management and analysis, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited only to those elements but may include other elements not expressly listed or inherent to such a process, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification, and all such embodiments are intended to be included within the scope of that term or terms. Language designating such non-limiting examples and illustrations includes, but is not limited to: “for example,” “for instance,” “e.g.,” “in one embodiment”.

In an embodiment of the present invention, a research and publication platform that embeds an automated system for variant annotation, interpretation, and integration directly into the publication process is provided. The invention provides a semantic annotation platform that accepts textual manuscripts and specialized biomedical data formats, such as DICOM, VCF, NWB, HPC logs, etc., and uses advanced NLP, AI-based annotation, and UMLS expansions, etc. to produce a machine-readable, semantically linked final publication in real time manner.

The semantic annotation platform comprises a user interface to receive at least one textual manuscript file and at least one biomedical data file. The at least one biomedical data file is selected from the group consisting of imaging data (DICOM file), genomic variant data (VCF file), electrophysiological data (NWB file), or other non-textual data (eg, CSV or custom format). The manuscript file is a textual file and can be in a Word, PDF, or LaTeX format. The platform comprises an internal parser based on Natural Language Processing (NLP) that extracts textual content from the manuscript file and identifies sections, such as Title, Abstract, Methods, Results, Figures, References, etc. Each of the uploaded files is indexed in the backend database of the semantic annotation platform and assigned a unique identifier. The unique identifier is a DOI, handle, or some persistent ID.

In an embodiment of the present invention, the semantic automation platform is configured to ingest single cell RNA seq proteomic file in HUPO-PSI format.

The semantic annotation platform is configured to provide semantic annotation and metadata extraction for the uploaded files. The semantic annotation platform comprises a semantic annotation engine that runs over both the manuscript text and the raw data files. The semantic annotation engines utilize Natural Language Processing (NLP) to identify biomedical entities, such as genes, conditions, procedures, etc., and link them to the standardized ontologies, such as UMLS, SNOMED CT, RxNorm, etc.

For non-textual biomedical files, the semantic annotation engine examines file headers or structured attributes to extract relevant metadata, for eg, Patient ID from DICOM, variant info from a VCF, or electrode metadata from NWB. The extracted metadata is normalized into a machine-readable format (JSON, XML, RDF, or JSON-LD), containing references to ontology terms and other standard vocabularies.

The semantic annotation engine comprises a cross-ontology module that ensures consistent naming across different domains. For example, a certain gene variant might be mapped to the relevant concept in UMLS; a drug reference might be mapped to RxNorm. The result is a semantically enriched dataset that includes both textual references and structured annotations, preserving relationships between them.

In an embodiment of the present invention, the semantic annotation platform combines manuscript text with structured data references. The platform maintains an internal document model that merges the text of the manuscript with the embedded references to the processed data objects. Each figure, table, or mention in the text is tied to a record in the metadata database, so that the final layout “knows” which data points are relevant. In an HTML-based or JSON-based final output, each mention of a dataset is converted into a clickable link or interactive widget. This allows a mouse hover or a click event to reveal underlying metadata (e.g., patient demographics, date, acquisition parameters) or to display a pop-up with the raw data preview.

In an embodiment of the present invention, the semantic annotation platform stores each dataset version (raw, processed, final) with cryptographic hashes or timestamps. This ensures data integrity and traceability, so that the dataset is accessible, tamper-evident, and fully documented.

In an embodiment of the present invention, the semantic annotation platform generates the interactive output in the form of HTML, JSON, and Interactive Visualization components. The semantic annotation platform compiles all textual sections, figures, and data references into multiple output formats, such as HTML for an interactive web-based publication, PDF for a static version, JSON or JSON-LD for machine-readable, containing ontological mapping and references. The structured metadata (eg, Data IDs, ontology codes) is embedded as microdata or linked data in the HTML output.

Interactive Visualization Components ensures that the output file when rendered in a browser or in interactive PDF file, each figure or data reference is able to show features, such as drill-down to raw data, annotations and QC flags, persistent URLs or DOIs. The user is able to click a figure to see the original DICOM slice, VCF variant calls, or NWB time series. If the semantic annotation platform flagged any quality issues, such as missing metadata, suspicious manipulations, etc., these appear as alerts. The user is able to follow a link to a data repository or directly download to access the document.

In an embodiment of the present invention, the platform is designed to support the integration of a wide variety of biomedical sources, including Genomic Data (DNA sequences, gene expression levels, variant data, and other omics-related information), Clinical Data (Electronic health records (EHRs), patient demographics, diagnosis codes, clinical test results, and medical histories), Imaging Data (Medical images such as DICOM files, radiology reports, and associated metadata), Proteomic Data (Data from protein expression studies, mass spectrometry results, and other protein-level assays).

In an embodiment of the present invention, a core feature of the platform is its use of semantic annotation. The system employs established biomedical ontologies, such as:

1. Gene Ontology (GO) for annotating gene functions and processes.

2. Unified Medical Language System (UMLS) for clinical terms and concepts.

3. Value Set Authority Center (VSAC), RxNorm for drug-related terms, and SNOMED CT codes for clinical concepts.

5. RadLex for radiology and medical imaging terminology.

The platform automates the process of applying these ontologies to the integrated data, ensuring that the data is semantically enriched. Each data element is annotated with appropriate terms and relationships from the relevant ontology, providing deeper insights and making the data easier to search, interpret, and link across datasets. The metadata scheme is designed to support a wide range of biomedical data types, including observational datasets, clinical trials, and imaging data. By leveraging VSAC and RxNorm for pharmaceutical terms and interventions, the platform ensures comprehensive tagging of all key data elements, improving interoperability and enabling advanced data analytics. Incorporating these UMLS Terminology Services also impacts the submission and review workflows on the platform. Metadata generation is automated during the document upload process, where key terms in manuscripts are identified, tagged, and mapped to appropriate UMLS, VSAC, RxNorm, and SNOMED CT codes. This automation extends to retrospective tagging of legacy content, ensuring that older studies meet current metadata standards for interoperability.

In one embodiment of the present invention, the metadata for figures, tables, and supplementary materials is enriched with NBO, NWB (Neurodata Without Borders), UMLS IDs, and other relevant standards to support cross-study analyses and interoperability.

The platform provides a guided interface for authors and reviewers to validate and refine metadata annotations, promoting user engagement and ensuring the highest quality of metadata. Furthermore, the metadata generated is seamlessly integrated with journal management workflows, supporting peer review, citation management, and enhancing discoverability through optimized metadata outputs, such as XML and JSON formats.

The platform stores the data in a centralized repository or database, once the data is integrated and semantically annotated. This repository allows users to query and analyze the data based on ontologically enriched attributes. The platform includes a query interface that supports both keyword searches and complex queries based on semantic relationships.

The platform enables the publication and sharing of integrated, semantically annotated biomedical data. It supports the following features:

2. Standardized Data Formats: Data is published in formats such as RDF (Resource Description Framework), JSON-LD, and OWL (Web Ontology Language), ensuring compatibility with other biomedical data repositories and tools.

3. Open Access Publication: Data can be published to public repositories or shared within specific research communities, following open-access principles.

4. Data Provenance and Versioning: Each data entry is associated with provenance information to track its origin, transformations, and updates. The platform also includes a version control system to track changes to the data over time.

5. Interoperability: The platform supports common data-sharing protocols such as FHIR (Fast Healthcare Interoperability Resources) for clinical data and HL7 for medical information exchange.

In an embodiment, the platform aligns with FAIR (Findable, Accessible, Interoperable, Reusable) principles by automating metadata structuring, reducing manual work, and making data easier for computational tools to process. The platform provides an integrated system within the publication platform that automatically formats, stores, and indexes genomic data in FAIR-compliant tables upon manuscript submission, promoting data discoverability and reusability in clinical settings. The platform achieves the FAIR principle by automatically indexing the published document with embedded metadata (Findable), Data for the published document is stored on a stored database/repository with DOIs/persistent identifiers (Accessible), using standard schemas and machine-readable formats, such as JSON, RDF, DICOM for imaging (Interoperable), detailed metadata, and licensing (Reusable). The semantic annotation platform incorporates features to comply with FAIR principles of NIH DMS Policy, such as:

3. Findable: Each dataset is assigned a unique, trackable identifier and embedded in the final document with semantic markup;

4. Accessible: The semantic annotation platform either hosts the data or links it to a recognized public repository, and relevant credentials or access conditions are included;

5. Interoperable: Ontologies (UMLS, SNOMED, etc.) ensure the data and metadata can be understood and integrated by other systems;

6. Reusable: The machine-readable metadata (JSON, RDF) plus persistent IDs enable other researchers to re-run analyses or re-check results.

In an embodiment of the present invention, the platform incorporates robust security features to ensure the privacy and integrity of sensitive biomedical data, particularly clinical data, such as Data Encryption (Both at rest and during transmission); Role-Based Access Control (Ensures that only authorized users can access or modify specific data); and Compliance with Data Protection Regulations. The platform complies with standards such as HIPAA (Health Insurance Portability and Accountability Act) and GDPR (General Data Protection Regulation) to protect patient privacy.

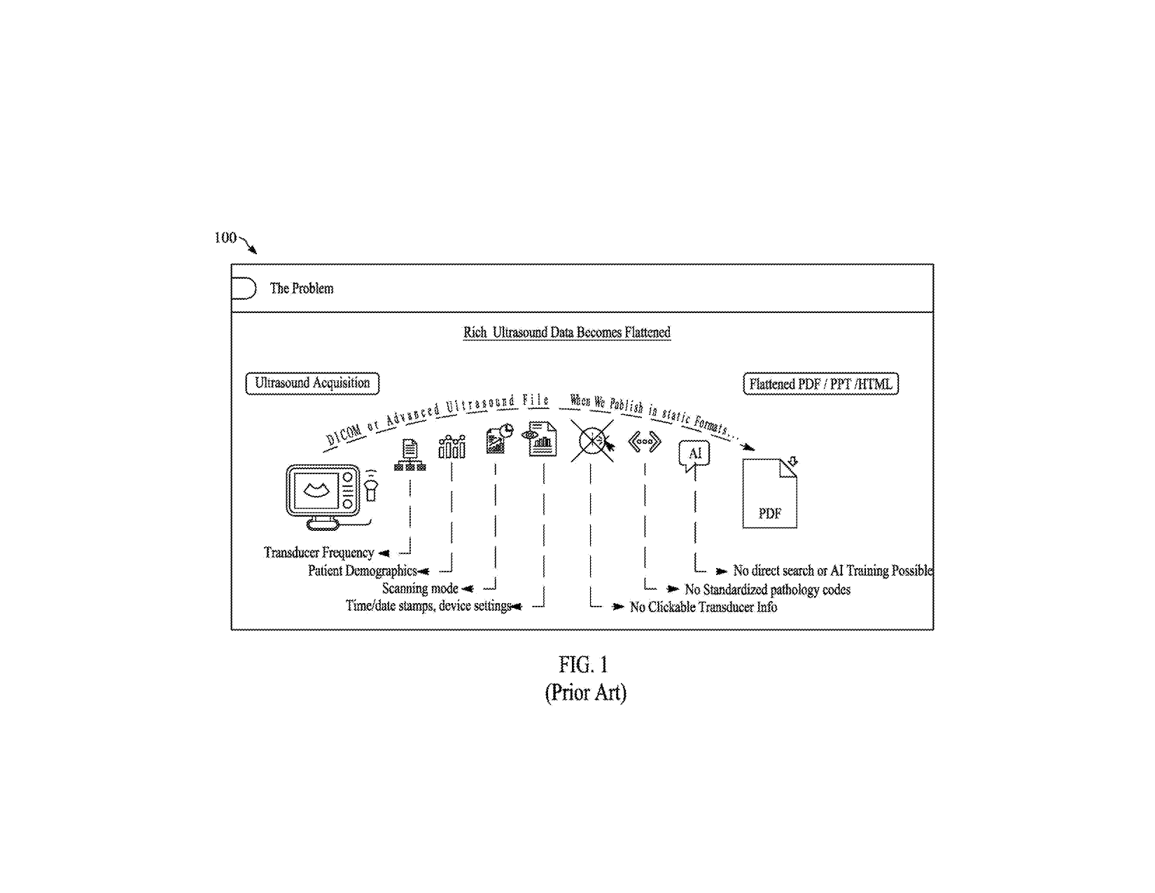

FIG. 1 shows a prior art publication system 100 illustrating the problem of data flattening in conventional research publication workflows. The prior art publication system 100 depicts an ultrasound acquisition process on the left side, where a DICOM or Advanced Ultrasound File contains rich data, including transducer frequency, patient demographics, scanning mode, time/date stamps, and device settings. The prior art publication system 100 demonstrates that when this rich ultrasound data is published in static formats, the data becomes flattened. The prior art publication system 100 shows various data elements, including an ultrasound device, patient information icons, scanning equipment representations, location markers, and imaging symbols that flow through the publication process. The prior art publication system 100 illustrates that when published in static formats such as flattened PDF, PPT, or HTML, the output loses interactivity and machine-readability. The prior art publication system 100 indicates that the resulting flattened output suffers from multiple limitations, including no direct search or AI training possible, no standardized pathology codes, and no clickable transducer information. This outdated publication model significantly impedes the ability of clinicians and researchers to leverage biomedical data insights, as any attempt to use this information requires laborious re-extraction, re-annotation, and reformatting of data that was already standardized at earlier stages.

In an embodiment, the present invention provides a semantic annotation platform for publishing research files that is capable of ingesting multiple file types, such as VCF, DICOM, NWB, HPC logs, etc., and embed them into a single interactive, machine-readable publication in real time.

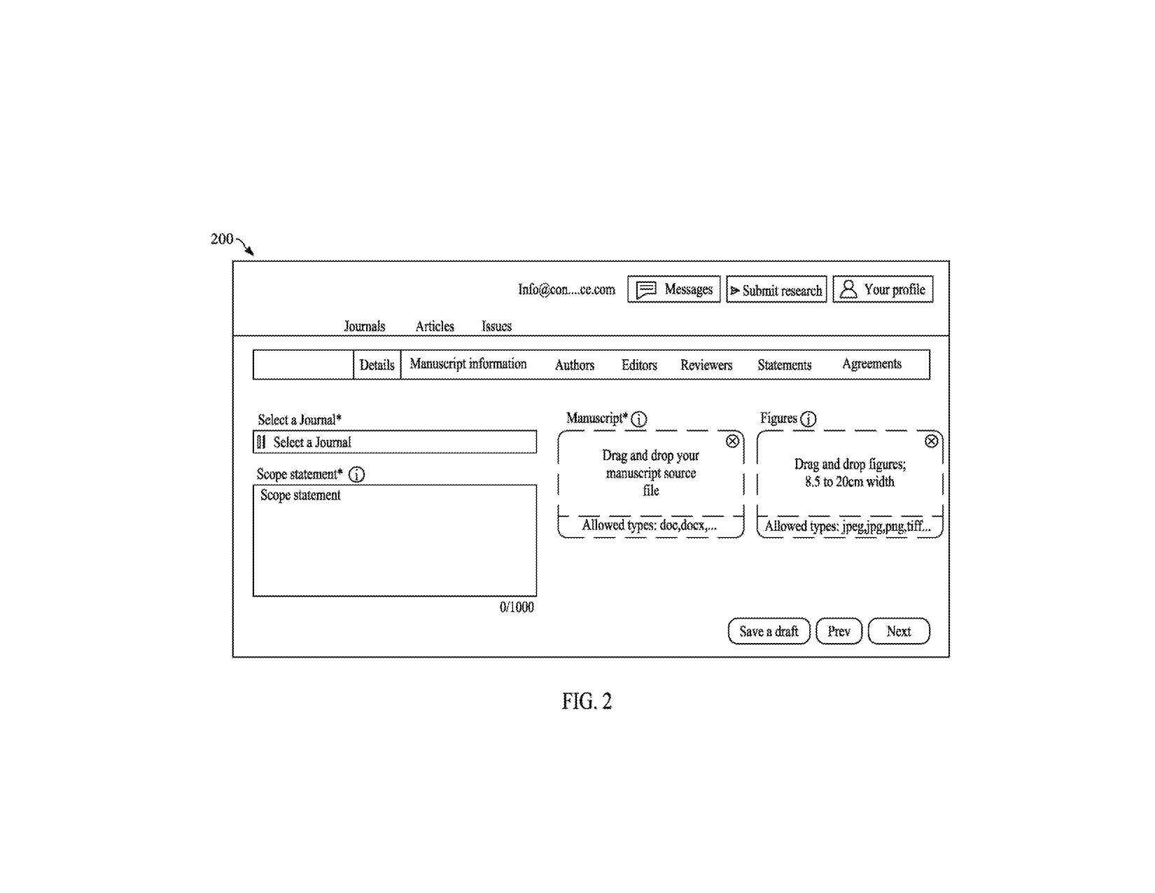

FIG. 2 illustrates a user interface 200 for uploading a document to the semantic annotation platform, in accordance with an embodiment of the present invention. The user interface 200 displays a navigation bar at the top containing menu options including Journals, Articles, Issues, and additional options for Submit research, Messages, and Your profile. Below the navigation bar, the user interface 200 presents a manuscript information section with multiple tabs, including Details, Manuscript information, Authors, Editors, Reviewers, Statements, and Agreements. The user interface 200 includes a journal selection field and a Scope statement field with a character counter. The user interface 200 provides a Manuscript upload area displaying instructions to drag and drop a manuscript source file with allowed types including doc and docx formats. Adjacent to the manuscript upload area, the user interface 200 includes a Figures upload area displaying instructions to drag and drop figures with specified dimensions and allowed types, including JPEG, JPG, PNG, and TIFF formats. The user interface 200 provides navigation controls at the bottom, including Save a draft, Prev, and Next buttons for managing the submission workflow. The user interface 200 provides a structured metadata intake system aligned with NIH and FAIR standards. The platform facilitates document uploads in multiple formats, automatically parses and classifies manuscript content into core sections such as Introduction, Methods, and Results, and recognizes flexible section variations. The platform detects figures, tables, and supplementary items and organizes them as distinct entities with contextual linkage preserved to enhance modularity. The platform detects VCF files, DICOM/ultrasound files, and NWB files, and annotates them with metadata from different sources, and provides contextual linkage to the files. The platform integrates with UMLS and utilizes NLP to enable precise metadata structuring, interoperability, and readiness for AI-driven annotation.

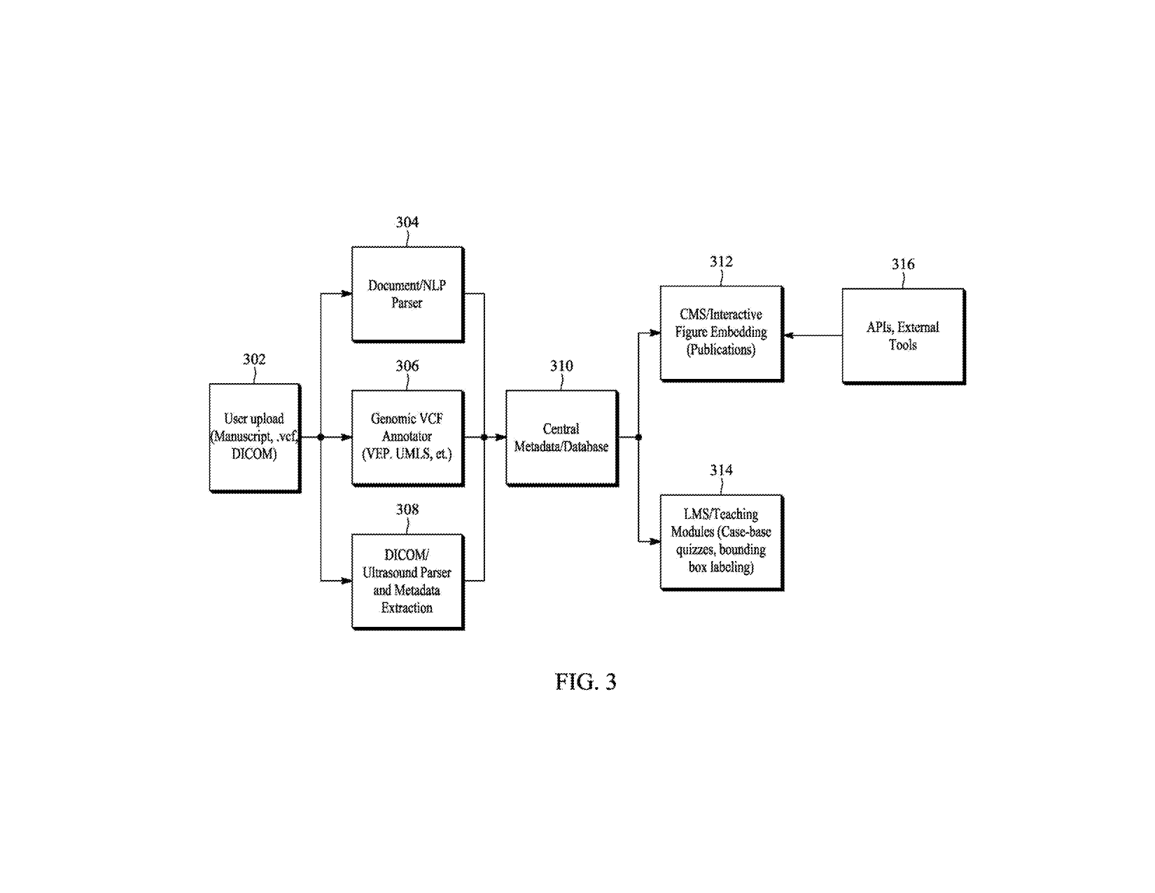

FIG. 3 is a block diagram showing a process flow for automated publication of heterogeneous biomedical data, in accordance with an embodiment of the present invention. The process flow begins with a user upload 302, which receives manuscript files and biomedical data files, including manuscripts, VCF files, and DICOM files. The user upload 202 connects to three parallel processing modules.

A document/NLP parser 304 receives and processes textual manuscript content using Natural Language Processing algorithms to extract structured information. The document/NLP parser 304 analyzes the text to identify key information such as patient details, research study data, diagnostic results, clinical notes, and medical terms. The document/NLP parser 304 identifies sections such as Title, Abstract, Methods, Results, Figures, and References.

A genomic VCF annotator 306 processes genomic variant data from VCF files. VCF files typically contain information such as genomic variants (SNPs, insertions, deletions), sample metadata (patient ID, sequencing method, read depth, quality scores), and functional annotations. The genomic VCF annotator 306 ingests these VCF files, extracts relevant metadata including variant information and sample details, and annotates variants using tools such as VEP (Variant Effect Predictor). The annotated variants are enriched with synonyms and medical codes from UMLS, VSAC, SNOMED CT, and RxNorm.

A DICOM/ultrasound parser 308 handles medical imaging data. DICOM files typically contain metadata such as patient ID, study description, image acquisition parameters, transducer frequency, scanning mode, time/date stamps, and device settings. The DICOM/ultrasound parser 308 extracts this metadata and processes images for integration with the manuscript content.

Each of the three processing modules connects to a central metadata/database 310, which serves as a unified repository for storing and indexing the processed and annotated data from all sources. The central metadata/database 310 combines the textual metadata from the manuscript with the structured metadata from the VCF and DICOM files into an integrated data model. Each uploaded file is indexed and assigned a unique identifier such as a DOI, handle, or persistent ID.

The central metadata/database 310 provides data to two output systems. A CMS/interactive figure embedding 312 utilizes the stored data to facilitate the creation, management, and publication of interactive figures and data visualizations within publications. Interactive figures enhance reader engagement by allowing them to interact with the data, explore different variables, or adjust parameters in real-time. An LMS/teaching modules 314 uses the stored data to create educational content, deliver, manage, and track educational activities. It enables instructors to create and organize teaching modules, including multimedia resources, quizzes, assignments, and discussions.

In an embodiment, the platform further comprises API’s external Tools 316 that provide APIs and export functionality support that provide seamless integration with external databases and tools, enabling broader applications such as clinical analyses and cross-disciplinary research.

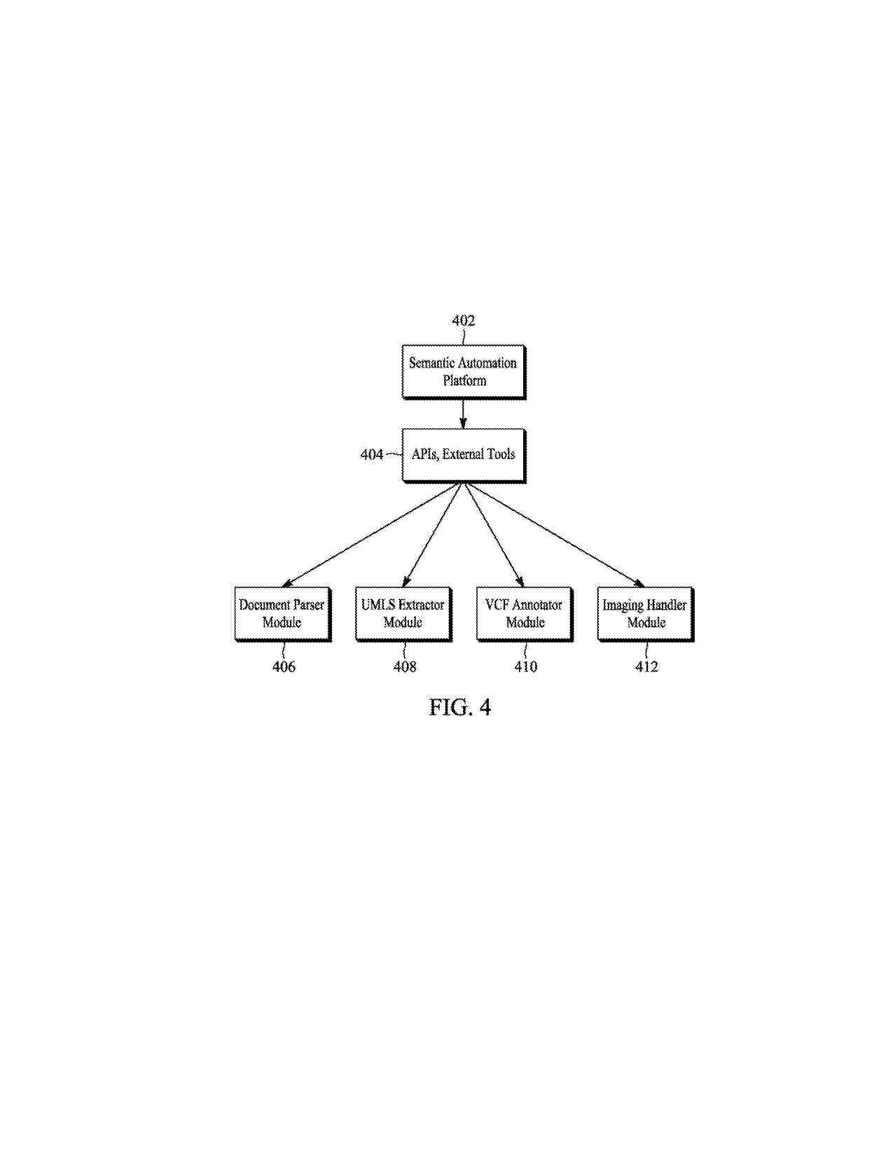

FIG. 4 is a block diagram showing an architecture of a semantic annotation platform for the automated publication of heterogeneous biomedical data, in accordance with an embodiment of the present invention. The architecture comprises a semantic annotation platform 402 positioned at the top of the hierarchy, which serves as the central processing system for annotating and integrating diverse biomedical data types. The semantic annotation platform 402 connects to APIs and external tools 404, which act as an interface layer that handles requests from external clients and routes them to appropriate processing modules. The APIs and external tools 404 act as a reverse proxy and provide functionality support for seamless integration with external databases and tools, enabling broader applications such as clinical analyses and cross-disciplinary research. Below the APIs and external tools 404, four specialized processing modules are arranged. The semantic annotation platform 402 comprises a document parser module 406, a UMLS extractor module 408, a VCF annotator module 410, and an imaging handler module 412. The modules interact through the APIs and external tools 404 to enable comprehensive analysis, where the document parser module 406 extracts textual information, the UMLS extractor module 408 enriches parsed text with standardized concepts, the VCF annotator module 410 processes genomic variant data, and the imaging handler module 412 processes medical images while linking them to clinical and genomic data for integrated analysis.

### Document Parser Module

The Document Parser module 406 is responsible for extracting and structuring information from textual documents (e.g., clinical notes, research papers, medical reports). The Document Parser module 406 utilizes Natural Language Processing (NLP) techniques to process unstructured text and annotate it with relevant medical concepts. The Document Parser module 406 is responsible for extracting text from various document formats, such as PDF, Word, etc. (Text Extraction); Identifying key biomedical entities such as diseases, drugs, proteins, symptoms, and clinical terms using pre-trained models, such as BioBERT, Med7 (Named Entity Recognition); Linking extracted entities to authoritative biomedical ontologies and knowledge bases, such as UMLS, VSAC, SNOMED CT (Entity Linking), RxNorm; Tagging extracted entities with metadata (e.g., disease names, treatment regimens) for further analysis and querying (Metadata Tagging); Standardizing clinical terminology to align with ontologies and reduce ambiguity in later steps (Text Normalization). The Document Parser module 406 processes the input with unstructured text (such as clinical notes, and research papers) and generates output with structured annotations (e.g., disease names, drug mentions) linked to relevant ontologies, standardized for further analysis.

### UMLS Extractor Module

The UMLS (Unified Medical Language System) Extractor module 408 is responsible for enriching textual data with standardized medical concepts from the UMLS Metathesaurus, which provides mappings to multiple healthcare terminologies. The UMLS Extractor module 408 is responsible for detecting and categorizing concepts such as diseases, procedures, drugs, genes, and other medical entities in the text (Concept Recognition); Mapping identified entities to UMLS Concept Unique Identifiers (CUIs), which are standardized identifiers in the UMLS system (UMLS Concept Mapping); Resolving synonyms, abbreviations, and different clinical terminologies to a single, standardized UMLS CUI (Semantic Normalization); annotating clinical text or research documents, providing a semantic layer of standardized concepts (Integration). The UMLS Extractor module 408 processes the input with text data from the Document parser module 406 or raw clinical documents and generates output with UMLS Concept Unique Identifiers (CUI) mapped to entities in the document (e.g., CUI for disease, drug) for further analysis.

### VCF Annotator Module

The VCF Annotator module 410 processes genomic data in the Variant Call Format (VCF) and annotates the genetic variants with additional information, such as gene associations, functional effects, and clinical significance. The VCF Annotator module 410 is responsible for: reading VCF files, which contain genetic variants (e.g., SNPs, indels) along with metadata (e.g., sample IDs, quality scores); Annotating variants with biological significance, using databases like ClinVar, dbSNP, and Ensembl to associate variants with diseases, gene functions, and known clinical interpretations; Predicting the functional consequences of variants (e.g., missense, synonymous mutations) using tools like SIFT, PolyPhen, or CADD; Mapping variants to known genes, signaling pathways, and biological processes. The VCF Annotator module 410 generates a comprehensive annotated VCF file that includes detailed metadata for each variant.

### Imaging Handler Module

The Imaging Handler module 412 is responsible for processing and annotating medical imaging data (e.g., DICOM files) and linking these images to clinical and genomic metadata. The Imaging Handler module 412 integrates medical images with structured metadata, allowing users to associate visual data with clinical conditions and genomic variants. The Imaging Handler Module 412: Supports various medical imaging formats (e.g., DICOM, NIfTI), processing image metadata such as acquisition parameters, modality (e.g., MRI, CT scan), and patient details; Extracts metadata from image headers, such as patient ID, study description, physician, and acquisition date; Processes images for feature extraction (e.g., segmenting tumors in MRI scans, detecting lesions) using image recognition models or machine learning techniques; Annotates images with relevant clinical and genomic data, linking image findings to patient diagnoses, treatments, or genomic variants; Links images to the underlying clinical data, genomic information (e.g., from the VCF Annotator), and textual annotations (e.g., from the Document Parser) for comprehensive analysis.

In an embodiment of the present invention, different modules of the semantic annotation platform 402 interact with each other to publish heterogeneous biomedical data. The document parser module 406 parses a clinical or research document, extracting textual information like diseases, treatments, and biomarkers. The UMLS Extractor module 408 enriches the parsed text with standardized medical concepts from UMLS, SNOMED CT, VSAC, RxNorm, etc., linking the extracted terms to unique identifiers (CUI). The VCF Annotator module 410 processes the genomic data from VCF files, annotating the variants with functional and clinical information. The Imaging Handler module 412 processes medical images (e.g., DICOM) and extracts metadata (e.g., patient details, imaging features). It links the images to clinical and genomic data for integrated analysis.

In an embodiment of the present invention, the semantic annotation platform 402 provides APIs and External tools 404 that provide functionality support for seamless integration with external databases and tools. The API Gateway acts as a reverse proxy that handles requests from clients (e.g., web applications and other services). It routes the requests to the appropriate module and aggregates responses as necessary.

In an embodiment of the present invention, the semantic annotation platform has a feature of a manuscript submission and peer review tracking system used by journals and publishers. The platform facilitates author submissions, assigns reviewers, manages revisions, and guides articles through the editorial decision-making process.

In an embodiment of the present invention, the semantic annotation platform utilizes a distributed micro-services architecture in which an Ontology Alignment Service (OAS) runs in parallel with the main annotation pipeline. The OAS takes as input every biomedical term or concept extracted from the document parser module 406. The OAS calls out multiple reference endpoints, such as UMLS Metathesaurus for general medical concepts, VSAC for value sets related to lab results or medications, RxNorm for drug information, SNOMED CT for clinical diagnoses and procedures, NBO for behavioral concepts, NWB standards for neurophysiological data structures. Each ontology returns potential matches with a “confidence” or similarity metric. The OAS aggregates these metrics, then applies an ensemble method, such as random forest or gradient-boosted trees trained on previously validated mapping to select the best fit. If two ontologies claim the same textual label but yield contradictory definitions, the OAS flags the conflict. A local resolution process attempts to unify them under a “parent” concept that best captures the meaning. If still unresolved, the system logs a “require user review” notice.

Before finalizing a mapping, the OAS consults metadata about the experiment or publication, such as Domain type (clinical vs. behavioral), Data modality (MRI vs genotyping), and Study design (human trial vs rodent model). This context can tip the scale towards one ontology’s concept over another. For instance, a term referencing “dosage” might lean toward an RxNorm mapping, while the same term in a neuro experiment might map to NBO if it is about behavior measurement.

The semantic annotation platform dynamically “creates” crosswalk rules whenever it encounters new terms or metadata contexts, instead of relying on static spreadsheets or manual crosswalk documents. The OAS keeps an internal “hierarchical dictionary”, updated daily, that merges shared synonyms and references from multiple ontologies. This ensures that each concept is placed in the correct semantic position across different ontological trees. All decisions, automated or user-approved, are stored in a version-controlled knowledge graph. Over time, the system learns to avoid repeating the same mistakes, significantly reducing manual curation.

The semantic annotation platform of the present invention merges research ontologies (NWB for electrophysiological recordings) with clinical ontologies (SNOMED, UMLS), creating a single integrated knowledge graph that covers everything from gene variants to lab protocols, imaging scans, and behavioral endpoints. The real-time feedback and machine learning-based resolution go beyond simple look-up tables, allowing the system to evolve as new terms and ontologies appear. The approach ensures multi-domain interoperability, as the platform simultaneously addresses clinical, behavioral, and genomic data.

In an embodiment of the present invention, a computer-vision-assisted data capture (CVADC) engine is integrated with the semantic annotation platform. The computer-vision-assisted data capture engine captures raw data (images, videos) and immediately applies cryptographic hashing, advanced QC (e.g., splicing or duplication checks), and metadata tagging. Storing and publishing these hashes and metadata ensures that reviewers or subsequent readers can verify a given figure truly corresponds to its original, unmanipulated file. The CVADC engine can automatically embed or generate metadata that describes each experiment, such as sample IDs, lot numbers, chemical reagents, acquisition parameters, time stamps, etc., in a standardized format (JSON, XML), so that each raw data file is tightly coupled with its experimental context. Each raw image or dataset is automatically hashed upon ingestion. The hash can be stored in a local or remote ledger. If the researcher processes the image further (Normalizing intensities or adjusting brightness), the CVADC engine tracks these changes as distinct “versions” that reference the parent raw data.

### SEMANTIC PROCUREMENT AND REFERRAL SUBSYSTEM

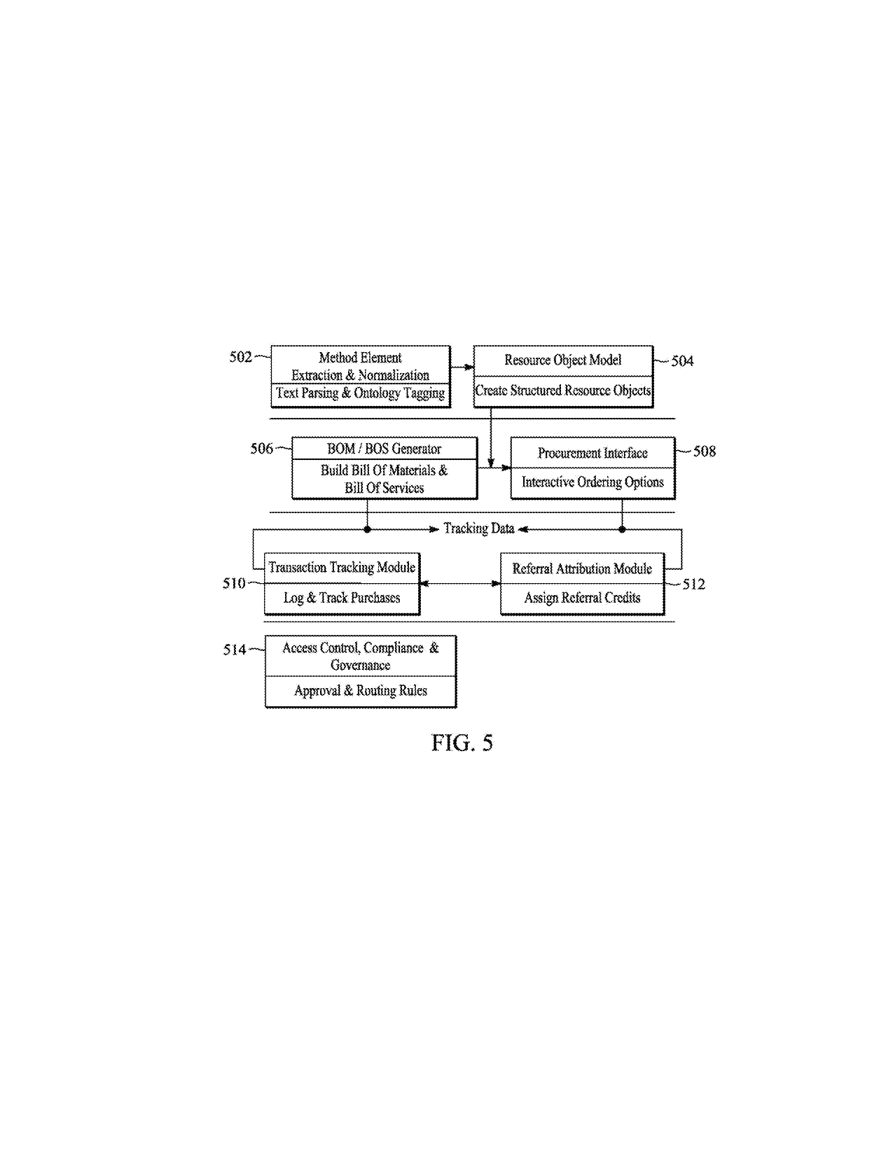

In an embodiment of the present invention, the semantic annotation platform includes a semantic procurement and referral subsystem that transforms Methods sections and other protocol elements into machine-actionable procurement interfaces. FIG. 5 illustrates a flow diagram showing the semantic procurement and referral subsystem. This subsystem enables the platform to function as an e-commerce and referral mechanism for sourcing the materials, instruments, software, datasets, and services required to replicate published research.

### Method Element Extraction and Normalization 502

The semantic procurement subsystem comprises a method element extraction module that parses Methods sections into discrete method steps and resource elements. Each method step represents a protocol action, while each resource element represents an input, tool, or service required to perform that action. The parsing utilizes natural language processing and pattern recognition to identify resource mentions within unstructured prose.

Each identified resource is tagged with ontology identifiers from standardized vocabularies such as UMLS, SNOMED CT, and domain-specific ontologies, as well as commercial identifiers including SKU, catalog number, vendor ID, software version, license type, and dataset accession number. The system supports disambiguation using AI-based entity resolution combined with user verification interfaces, allowing authors and editors to confirm or correct resource identifications.

### Resource Object Model 504

The semantic procurement subsystem comprises a resource resolution module that defines structured "Resource Objects" and "Method Step Objects" that capture comprehensive information about each resource and its context within the experimental workflow. A Resource Object may include fields such as: resource_name, vendor, catalog_number, SKU, lot_number, version, concentration, unit, quantity, acceptable_substitutes, shipping_constraints, regulatory_flags, and institutional_contract_ID.

Each Resource Object is assigned a persistent identifier that enables tracking and linking across publications, procurement systems, and institutional databases. Resource Objects maintain linkage to their associated Method Step Objects, preserving context such as organism or model system, assay type, instrument settings, and experimental conditions. This contextual linkage ensures that procurement actions are informed by the specific experimental requirements.

### Procurement Endpoints and Actions

Each Resource Object may link to one or more procurement endpoints that enable direct action from within the publication. The semantic procurement subsystem comprises a procurement interface 508 that presents procurement endpoints as interactive elements within the rendered publication. Procurement endpoints may include: purchase now, request quote, institutional purchase requisition, license software, request dataset access, and schedule service (such as sequencing or imaging services).

The semantic procurement subsystem further comprises a BOM/BOS generator 506 that may generate a "shopping cart" interface that aggregates multiple Resource Objects for batch procurement. The BOM/BOS generator 506 may also generate a comprehensive "Bill of Materials" (BOM) listing all physical resources and a "Bill of Services" (BOS) listing all services, software licenses, and data access requirements. These BOM/BOS outputs may be exported in machine-readable formats such as JSON or XML for integration with institutional procurement systems.

### Referral Credit, Tracking, and Attribution

The semantic procurement subsystem comprises a transaction tracking module 510 and a referral attribution module 512 that generate referral links or referral identifiers embedded within the publication that track downstream procurement actions. When a reader initiates a purchase, license request, or data access request through the publication interface, the referral attribution module 512 may attribute that action back to the publication, author, institution, or platform.

These attribution events are recorded by the transaction tracking module 510 as "transaction metadata" that captures the relationship between the publication and the procurement action without requiring the platform to process payments directly. Transaction metadata may include: publication identifier, resource object identifier, action type, timestamp, user institution, and referral source. The system may optionally support revenue sharing, credits, coupons, or accounting reports based on recorded transaction metadata.

### Access Control, Compliance, and Governance 514

The semantic procurement subsystem incorporates safeguards to ensure ethical and compliant operation. The system may maintain conflict-of-interest disclosure metadata that identifies financial relationships between authors and vendors whose products are referenced in the publication. This disclosure information may be rendered alongside procurement interfaces to ensure transparency.

The semantic procurement subsystem may further comprise an institutional routing module that enforces institutional vendor restrictions and approval workflows, routing procurement requests through appropriate institutional channels based on the user's affiliation and the institution's procurement policies. Audit logs with cryptographic hashes may record all procurement actions and edits to Resource Objects, ensuring traceability and tamper-evidence.

In some embodiments, the platform may implement separation between editorial decision-making and commerce functions to address potential ethics concerns. Editorial workflows may proceed independently of procurement subsystem operations, ensuring that commercial considerations do not influence publication decisions.

### Version Pinning and Substitution Logic

In an embodiment of the present invention, the resource resolution module supports version pinning for software and datasets to ensure exact replication of published research. When a Resource Object references software, the system may capture and store the specific version number, build identifier, commit hash, or release date. For datasets, the system may record version identifiers, access dates, and checksums to ensure that subsequent users obtain the identical data used in the original study. Version pinning may be enforced at the time of manuscript submission, requiring authors to specify exact versions rather than generic references.

The resource resolution module may implement substitution logic using ontology-based equivalence when specified resources are unavailable. When a resource becomes discontinued, out of stock, or otherwise inaccessible, the system may query ontological relationships to identify functionally equivalent alternatives. Substitution candidates may be ranked based on semantic similarity, user ratings, citation frequency, or institutional preferences. The system may notify users when substitutions are proposed and require explicit approval before proceeding with the procurement of substitute resources. Substitution events may be logged as part of the transaction metadata to maintain provenance and enable analysis of substitution patterns.

### Replication Package Generation

In an embodiment of the present invention, the semantic procurement subsystem may generate downloadable replication packages that consolidate all information and resources required to reproduce a published study. A replication package may comprise the Bill of Materials, Bill of Services, detailed protocol steps extracted from the Methods section, dataset access links with authentication tokens or request workflows, software installation scripts or container definitions, instrument configuration files, and environmental parameters.

Replication packages may be exported in multiple formats, including JSON, XML, or PDF for human-readable summaries. The system may generate containerized environments such as Docker or Singularity containers that encapsulate software dependencies and configurations. Replication packages may include executable scripts that automate resource procurement, data retrieval, and environment setup. Each replication package may be assigned a persistent identifier and versioned independently of the publication, enabling tracking of package downloads and usage.

### Integrationwith Computer-Vision-Assisted Data Capture Engine

In an embodiment of the present invention, the semantic procurement subsystem integrates with the computer-vision-assisted data capture (CVADC) engine to automatically populate Resource Objects with metadata captured at the point of experimental data acquisition. When the CVADC engine captures images, videos, or sensor data, it may simultaneously record contextual metadata, including antibody lot numbers, reagent batch identifiers, instrument serial numbers, calibration dates, and acquisition parameters. This captured metadata may be automatically mapped to corresponding Resource Objects, ensuring that procurement specifications reflect the exact materials and conditions used in the experiment.

The integration between the CVADC engine and the procurement subsystem may enable fraud detection to trigger procurement verification workflows. When the CVADC engine detects anomalies such as image manipulation, metadata inconsistencies, or chain-of-custody violations, the system may flag associated Resource Objects for verification. Reviewers or editors may be prompted to confirm that referenced resources match the captured experimental conditions. This integration establishes a chain of custody from initial data capture through procurement, ensuring that published resource specifications accurately reflect experimental reality.

### Predictive Procurement and Resource Recommendations

In an embodiment of the present invention, the semantic procurement subsystem may incorporate machine learning models that predict resources a researcher may need based on their research profile, publication history, institutional affiliation, or current manuscript content. Predictive procurement models may analyze patterns across the platform's corpus of publications to identify commonly co-occurring resources, enabling proactive suggestions during manuscript preparation. The system may present predicted resources as recommendations that authors can accept, modify, or reject.

The semantic procurement subsystem may include a resource recommendation engine that suggests alternative or complementary resources based on study type, organism model, assay methodology, or research domain. Recommendations may be generated using collaborative filtering based on procurement patterns of similar researchers, content-based filtering based on semantic similarity of resource descriptions, or hybrid approaches combining multiple signals. The system may also support automated protocol optimization by analyzing successful replications and suggesting resource substitutions or methodological refinements that improve reproducibility or reduce costs.

### External System Integration

In an embodiment of the present invention, the semantic procurement subsystem may integrate with institutional Enterprise Resource Planning (ERP) and procurement systems such as SAP, Oracle, or Workday. Integration may enable automatic synchronization of vendor catalogs, contract pricing, and approval hierarchies. When a user initiates procurement from a publication, the system may route requests through the user's institutional procurement infrastructure, applying negotiated pricing and required approval workflows. Integration may be achieved through standardized APIs, file-based data exchange, or middleware connectors.

The semantic procurement subsystem may integrate with Electronic Lab Notebooks (ELNs) to enable bidirectional data flow between publications and ongoing research documentation. Resource Objects from publications may be imported into ELN experiments, while ELN entries may be exported to populate Methods sections during manuscript preparation. The system may also integrate with Laboratory Information Management Systems (LIMS) to track resource inventory, consumption, and reordering.

Integration with grant management systems may enable linking of procurement actions to funded projects, facilitating budget tracking and compliance reporting for funding agencies.

### Analytics and Reporting

In an embodiment of the present invention, the semantic procurement subsystem may generate analytics and reports based on procurement activity, referral events, and resource usage patterns. Reproducibility metrics may track how frequently studies are replicated using the generated Bills of Materials, measuring the time between publication and first replication attempt, success rates of replication efforts, and resource substitution frequencies. Resource popularity analytics may identify which reagents, instruments, software tools, or services are most commonly referenced and procured across the platform.

The system may provide author dashboards displaying procurement activity initiated from their publications, referral credits earned, and reproducibility metrics for their studies. Institutional dashboards may aggregate procurement data across affiliated researchers, enabling analysis of spending patterns, vendor relationships, and compliance with procurement policies. Journal dashboards may display platform-wide analytics, including resource trends, reproducibility indicators, and commerce activity. All analytics may be exported in standard formats for integration with external business intelligence tools. The transaction tracking module 510 may generate scheduled or on-demand reports summarizing referral activity, attribution events, and optional revenue sharing calculations.

### Security and Compliance

In an embodiment of the present invention, the semantic procurement subsystem may utilize blockchain or distributed ledger technology to create immutable records of procurement transactions, resource specifications, and attribution events. Each transaction may be cryptographically signed and appended to a distributed ledger, ensuring that procurement records cannot be altered or deleted after creation. Smart contracts may automate referral credit distribution, revenue sharing calculations, or compliance verification based on predefined rules encoded in the ledger.

The semantic procurement subsystem may implement compliance controls for regulations, including GDPR, HIPAA, and export control requirements. For procurement involving patient-derived materials, human tissue samples, or other regulated resources, the system may enforce consent verification, data protection requirements, and access restrictions based on user credentials and institutional agreements. Export control compliance may be enforced for regulated materials, equipment, or software by screening procurement requests against restricted party lists and requiring appropriate licenses or certifications before completing transactions. Audit trails with cryptographic integrity verification may support regulatory inspections and compliance audits.

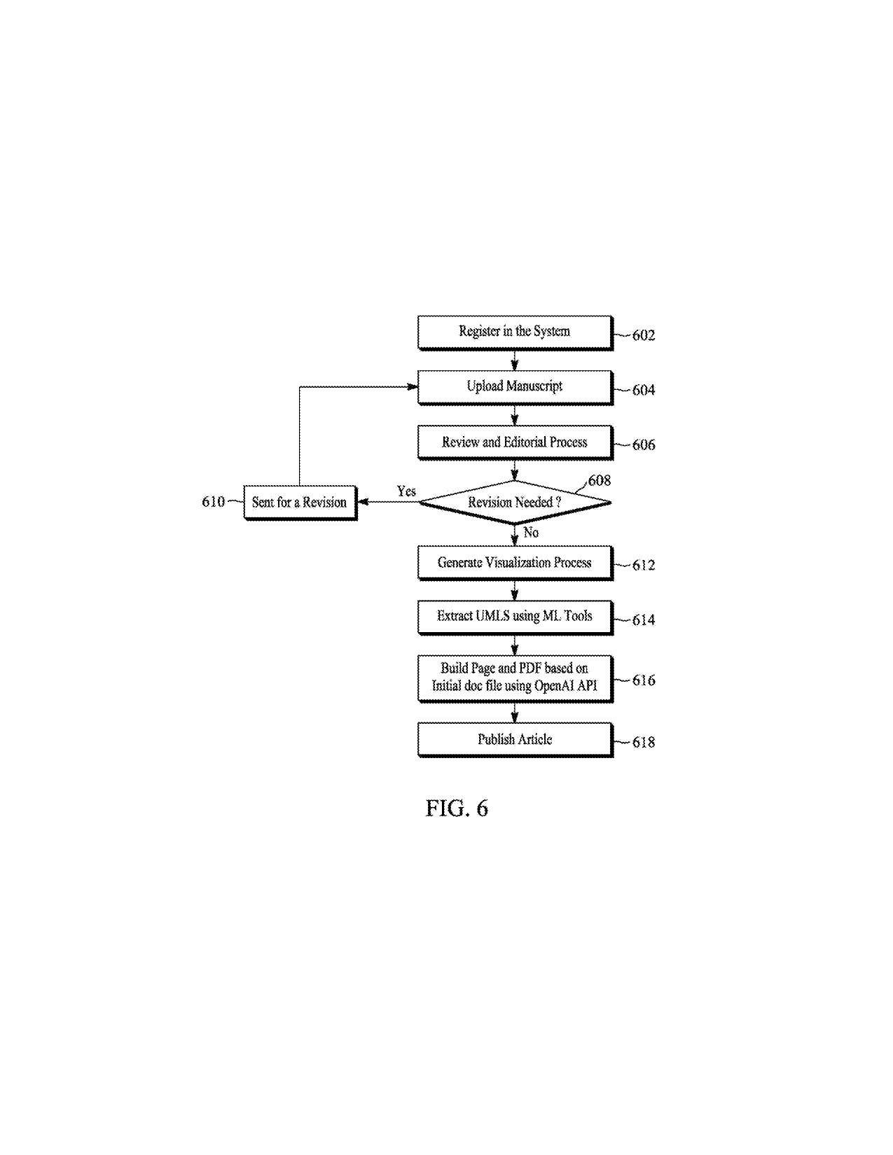

FIG. 6 is a flow diagram showing the manuscript submission and peer review tracking integrated in the semantic annotation platform, in accordance with an embodiment of the present invention. In the first step 602, a user registers on the platform. In the next step 604, the user uploads a manuscript. In the next step 606, the review and editorial process takes place. In the next step 608, the platform queries whether a revision is needed for the submitted manuscript. If yes, then in step 610, the platform sends the manuscript for revision, and after revision, the revised manuscript is uploaded again. If the platform decides that revision is needed for the document, then the process follows to step 612, where the visualization process is generated. In step 614, the UMLS, VSAC, SNOMED CT, and RxNorm tags are extracted using the UMLS extractor. In the next step 616, the platform builds a page and a PDF based on the initial docs file using the OpenAI API. In the next step 618, the platform publishes the article.

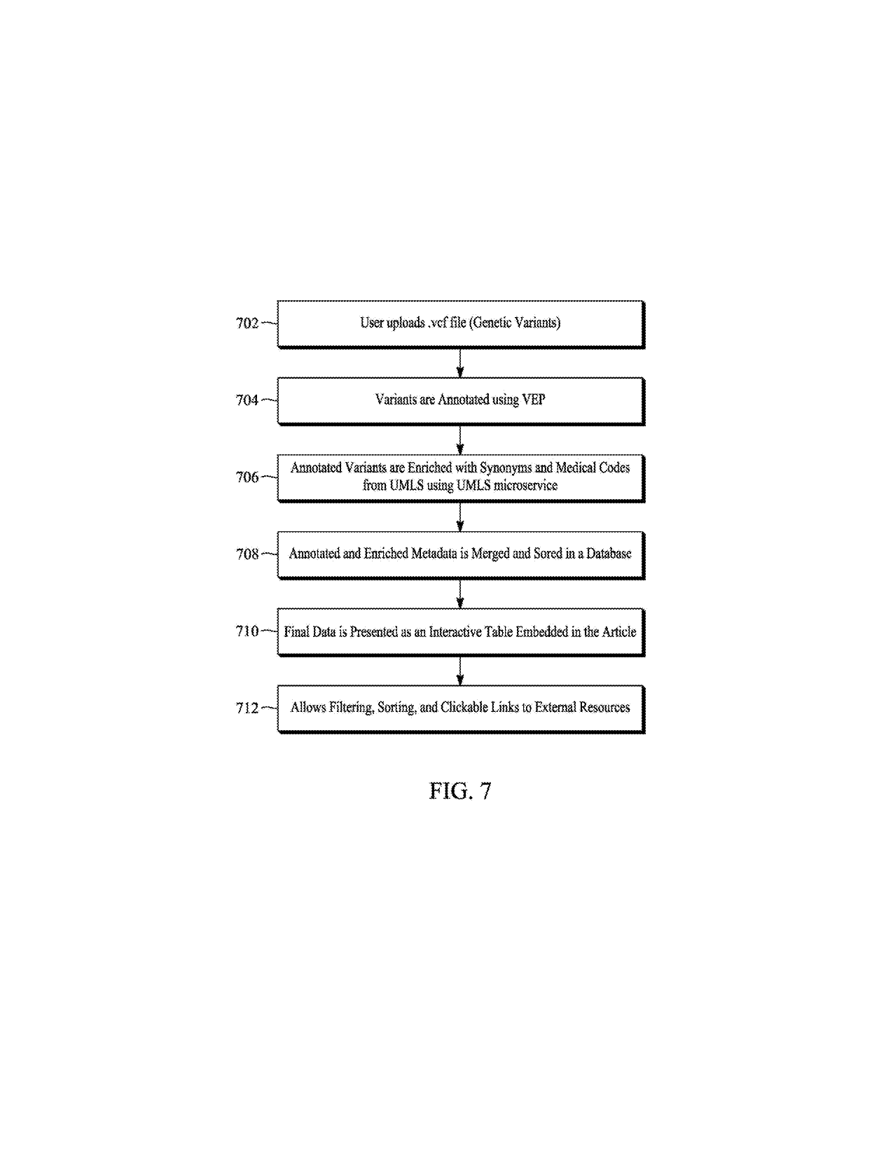

FIG. 7 shows a flow diagram illustrating a method for ingesting Variant Call Format (VCF) files and integrating metadata to support enhanced biomedical data management and analysis, in accordance with an embodiment of the present invention. The method ingests genomic data from VCF files, which represent variant information from DNA sequencing. The VCF files are annotated with VCF metadata that enables efficient search, visualization, and analysis, facilitating better decision-making in clinical and research contexts. In step 702, the user uploads a .vcf file containing the genomic variant along with the manuscript in the semantic annotation platform. In the next step 704, the VCF file is ingested, and variants are annotated using VEP. VCF files represent genetic variant data and typically contain information, such as Genomic variants (SNPs, insertions, deletions, and other sequence changes), sample metadata (Information about the sample, including patient ID, sequencing method, read depth, and quality scores), functional annotations (Predicted effects of variants on genes or proteins, based on databases like dbSNP, ClinVar, and others). The VCF files are ingested, and the relevant metadata is extracted, including variant information and sample details. This genomic data is then processed and converted into a standardized format, ensuring compatibility with the integrated dataset. In the next step 706, annotated variants are enriched with synonyms and medical codes from UMLS, VSAC, SNOMED CT, and RxNorm using the UMLS microservice. The metadata integration includes: Linking genomic variants to clinical data, enriching data with annotations (Adding relevant annotations from genomic databases to the integrated metadata, and cross-referencing textual and genomic data.

In the next step 708, the integrated metadata is indexed to support efficient querying, allowing researchers or clinicians to search across both the manuscript text and the genomic data for insights. The annotated and enriched metadata is merged and stored in the central repository or database.

In the next step 710, the final data is presented as an interactive table embedded in the article. The platform provides tools for visualizing the integrated data, such as Visualization of genomic data (Graphs and charts showing variant frequencies, distribution across populations, or functional impacts of variants), Visualization of clinical data (Patient cohort analysis, disease prevalence, and treatment outcomes associated with specific genetic variants), Custom dashboards (Allowing researchers and clinicians to create tailored views of the data that focus on relevant aspects of both the genomic and textual information).

In the next step 712, the method allows users to search and filter the integrated dataset. Users can perform queries that combine both textual and genomic data, such as: Finding research studies that include specific genetic variants, Searching clinical reports for patients with specific genetic conditions or variants, and Querying for associations between certain genes and diseases or treatments. The query system supports semantic searches based on the integrated metadata, allowing users to retrieve results that cross-reference textual and genomic information.

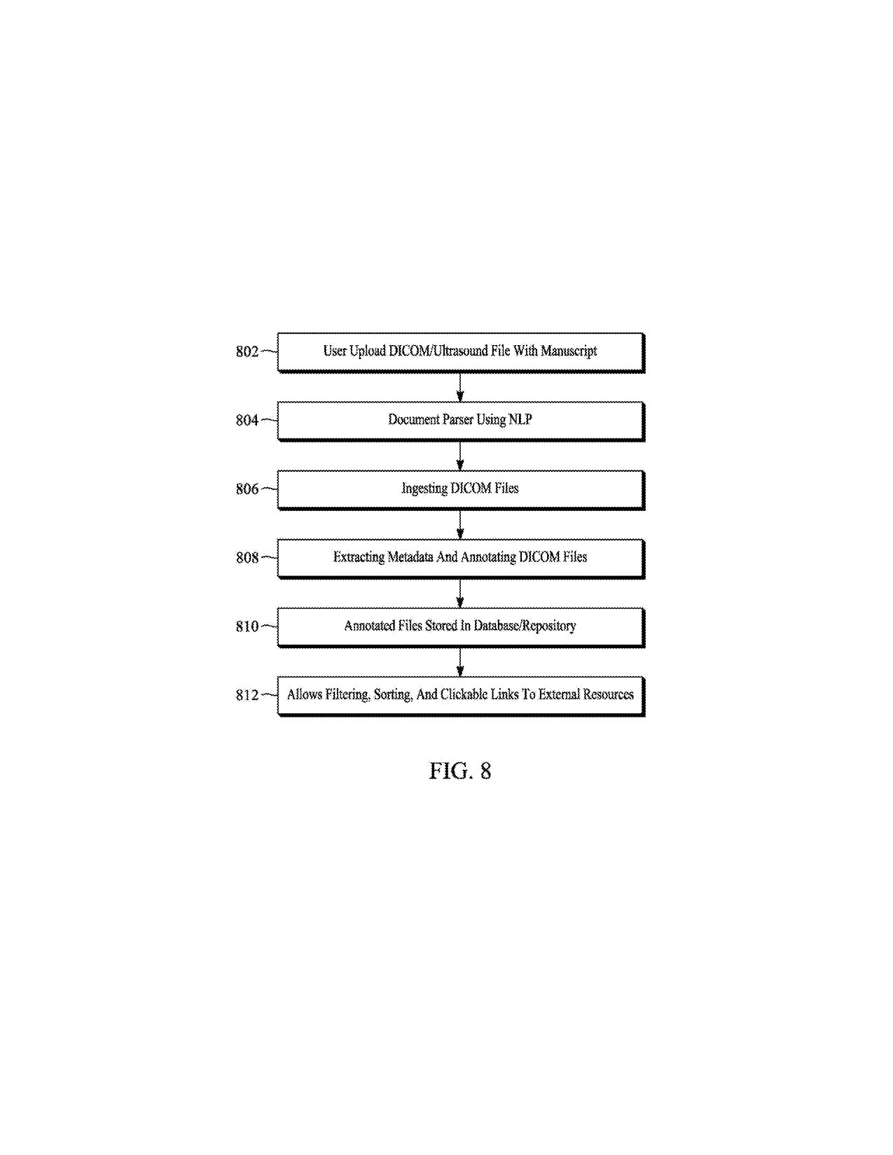

FIG. 8 shows a flow diagram illustrating a method for ingesting DICOM (Digital Imaging and Communications in Medicine) files and integrating relevant metadata to support enhanced biomedical data management and analysis, in accordance with an embodiment of the present invention. The platform utilizes natural language processing (NLP) techniques to extract structured information from textual documents, while also enabling the seamless extraction and organization of image and clinical data from DICOM files. The metadata from both the manuscripts and DICOM files are combined and indexed to create an integrated repository for easy access, analysis, and cross-referencing, facilitating clinical research, diagnostics, and data-driven decision-making. In step 802, the user uploads DICOM/ultrasound file containing images along with the manuscript in the semantic annotation platform. In the next step 804, the platform utilizes Natural Language Processing (NLP) algorithms to process and parse textual manuscripts. The NLP engine analyzes the text to identify key information such as patient details, research study data, diagnostic results, and clinical notes. The system can automatically identify and tag entities such as patient names, medical conditions, treatment plans, and other relevant terms, extracting structured data from unstructured content. Text normalization techniques are applied to standardize terminology and make the extracted data consistent.

In the next step 806, DICOM files are ingested. DICOM files typically contain metadata such as patient ID, study description, image acquisition parameters, and more. The platform extracts this metadata and stores it in a centralized database, associating the DICOM images with the corresponding clinical and research information. The images are processed and indexed for efficient retrieval.

In the next step 808, after parsing manuscripts and ingesting DICOM files, the platform integrates the extracted metadata into a unified data model. This includes combining the textual metadata from the manuscript (e.g., clinical information, research findings) with the structured metadata from the DICOM files (e.g., patient demographics, imaging data).

In the next step 810, the annotated file is stored in the central repository or database. The metadata is cross-referenced to create a comprehensive, integrated data repository that can be searched, analyzed, and visualized. The system may also apply machine learning techniques to correlate and link metadata from different sources, further enriching the dataset.

In the next step 812, the platform provides a powerful search engine that allows users to query the integrated dataset. Users can search for specific patient cases, clinical conditions, research studies, or DICOM images based on the metadata. The system supports advanced filtering, faceted search, and full-text search to enable precise and rapid information retrieval.

### USE CASE EXAMPLES

The following use case examples illustrate the end-to-end workflow of the semantic annotation platform from data upload through interactive publication and procurement.

### Genomics Study Use Case

In a first use case example, a researcher publishes a cancer genomics study that includes VCF files containing somatic mutation data from tumor samples. The researcher uploads a textual manuscript file describing the study methodology and findings, along with VCF files containing genomic variant data from The Cancer Genome Atlas (TCGA) or similar sources.

Upon upload, the semantic annotation platform ingests the manuscript through the document/NLP parser 304, which identifies sections including Abstract, Methods, Results, and References. The platform simultaneously processes the VCF files through the genomic VCF annotator 306, which extracts variant information, including SNPs, insertions, and deletions, along with sample metadata such as patient identifiers, sequencing methods, and quality scores.

The VCF annotator module 410 annotates the variants using VEP (Variant Effect Predictor) to determine functional consequences such as missense mutations, frameshift variants, or splice site alterations. The UMLS extractor module 408 enriches the annotated variants with standardized medical codes from UMLS, VSAC, SNOMED CT, and RxNorm, linking gene names to canonical identifiers and associating variants with known disease associations from ClinVar.

The platform generates interactive variant tables embedded within the published article, allowing readers to sort variants by gene, consequence, or clinical significance. Each variant entry may include clickable links to external databases such as dbSNP, ClinVar, and Ensembl. The semantic annotation engine extracts structured metadata and incorporates it into a semantically enriched article layout that remains machine-readable and compliant with NIH DMS Policy requirements.

The semantic procurement subsystem parses the Methods section through the method element extraction module 502, identifying resources such as DNA extraction kits, library preparation kits, sequencing reagents, and bioinformatics software. The resource resolution module resolves each resource to a Resource Object containing canonical identifiers and commercial identifiers, including vendor names, catalog numbers, and software versions. For example, a reference to "Illumina TruSeq DNA Library Prep Kit" may be resolved to a Resource Object containing the vendor (Illumina), catalog number (FC-121-2001), and current pricing information.

The BOM/BOS generator 506 generates a Bill of Materials listing all physical resources required to reproduce the genomics study, including sequencing kits, library preparation reagents, and consumables. The procurement interface module 508 presents interactive procurement endpoints within the publication, enabling readers to purchase reagents directly, request institutional quotes, or add items to a shopping cart for batch procurement. The transaction tracking module 510 records referral metadata when procurement actions are initiated, attributing purchases back to the publication and author.

### Medical Imaging Study Use Case

In a second use case example, a radiologist publishes an ultrasound imaging study examining diagnostic accuracy for a specific clinical condition. The radiologist uploads a textual manuscript along with DICOM files containing ultrasound images and associated metadata.

The semantic annotation platform ingests the DICOM files through the DICOM/ultrasound parser 308 and the imaging handler module 412. The platform extracts imaging metadata, including patient identifiers, study descriptions, image acquisition parameters, transducer frequency, scanning mode, time and date stamps, and device settings. This metadata is normalized into machine-readable formats and linked to the manuscript content.

The document parser module 406 processes the manuscript text, identifying clinical terms, anatomical references, and diagnostic criteria. The UMLS extractor module 408 enriches these terms with standardized codes from UMLS, SNOMED CT, and RadLex for radiology-specific terminology. The platform creates cross-references between textual descriptions and corresponding DICOM images, enabling readers to click on clinical findings and view the associated imaging data.

The published article presents ultrasound images as interactive figures rather than static images. Readers may hover over or click on image regions to view acquisition parameters, zoom into specific areas, or access the original DICOM data for independent analysis. The semantic annotation platform preserves the rich metadata that would otherwise be flattened in conventional PDF publications.

The semantic procurement subsystem identifies resources from the Methods section, including the ultrasound equipment model, transducer specifications, imaging software, and any contrast agents or consumables used. The resource resolution module creates Resource Objects for each identified resource, linking equipment references to manufacturer specifications and current product offerings. The BOM/BOS generator 506 generates a Bill of Services listing imaging equipment, calibration services, and software licenses required to replicate the imaging protocol.

The procurement interface module 508 enables readers to request quotes for imaging equipment, schedule equipment demonstrations, or inquire about service contracts. For consumables such as ultrasound gel or probe covers, direct purchase options may be presented. The institutional routing module may route equipment inquiries through institutional procurement channels, applying negotiated pricing from existing vendor contracts.

### Multi-Omics Study Use Case

In a third use case example, a researcher publishes a comprehensive multi-omics study combining genomic sequencing data, proteomic analysis, and medical imaging to characterize a disease phenotype. The researcher uploads a textual manuscript along with VCF files containing genomic variants, mass spectrometry data files in HUPO-PSI format containing proteomic results, and DICOM files containing relevant medical images.

The semantic annotation platform simultaneously processes all data types through the appropriate modules. The genomic VCF annotator 306 and VCF annotator module 410 process variant data and annotate mutations with functional predictions and clinical associations. The imaging handler module 412 extracts metadata from DICOM files and links imaging findings to clinical observations. The document parser module 406 and UMLS extractor module 408 process the manuscript text, identifying and standardizing biomedical terms across all data modalities.

The platform performs cross-ontology alignment to ensure consistent annotation across genomic, proteomic, and imaging data. For example, a gene identified in the VCF data may be linked to its protein product in the proteomic data and to imaging findings showing phenotypic manifestations. The Ontology Alignment Service runs in parallel with the annotation pipeline, querying multiple reference endpoints including UMLS Metathesaurus, Gene Ontology, and domain-specific ontologies to resolve and harmonize terms across modalities.

The published article presents an integrated view of multi-omics data, with interactive visualizations that allow readers to explore relationships between genomic variants, protein expression levels, and imaging findings. Readers may click on a genomic variant to see associated protein changes and corresponding imaging abnormalities. The machine-readable output includes JSON-LD representations with cross-references across all data types.

The semantic procurement subsystem generates a comprehensive Bill of Materials and Bill of Services covering all resources required to reproduce the multi-omics study. The method element extraction module 502 parses the Methods section to identify genomic sequencing reagents, mass spectrometry consumables, chromatography columns, imaging equipment, and analysis software. The resource resolution module creates Resource Objects for each resource category, capturing vendor information, catalog numbers, software versions, and instrument configurations.

The BOM/BOS generator 506 produces separate Bills of Materials for genomic, proteomic, and imaging components, as well as a consolidated bill covering all study resources. The system may generate a complete replication package comprising the Bills of Materials and Services, detailed protocol steps extracted from the Methods section, dataset access links with authentication workflows, software installation scripts, and instrument configuration files.

The procurement interface module 508 presents categorized procurement options, allowing readers to procure resources for specific experimental components or the entire study. The transaction tracking module 510 and referral attribution module 512 record all procurement actions, generating analytics on resource usage patterns and enabling author attribution for purchases initiated from the publication. The institutional routing module coordinates procurement across multiple vendor relationships, applying institutional preferences and approval workflows as appropriate.

This multi-omics use case demonstrates the full capability of the semantic annotation platform to handle heterogeneous biomedical data types, perform cross-modal annotation and linking, generate comprehensive procurement documentation, and enable end-to-end reproducibility from publication to resource acquisition.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A semantic annotation platform for research and publication, the platform comprising:
a user interface configured to receive at least one textual manuscript file and at least one biomedical data file, wherein the at least one biomedical data file is selected from the group consisting of DICOM imaging data, VCF genomic variant data, NWB electrophysiological data, and non-textual data;
a semantic annotation engine configured to extract structured metadata from the at least one biomedical data file by applying at least one standardized clinical or biomedical ontology;
wherein the platform incorporates said structured metadata into a semantically enriched article layout that remains machine-readable; and
wherein the platform produces an interactive publication output that simultaneously renders textual narrative and references to the structured biomedical data.

2. The semantic annotation platform of claim 1, wherein the interactive publication output is compliant with the NIH Data Management and Sharing Policy.

3. The semantic annotation platform of claim 1, wherein the semantic annotation engine is further configured to embed machine-readable metadata, assign persistent identifiers, and link data to at least one terminology selected from the group consisting of UMLS, VSAC, RxNorm, and SNOMED CT.

4. The semantic annotation platform of claim 1, wherein the semantic annotation engine uses natural language processing and pattern recognition algorithms to classify content into predefined sections and enhance metadata annotation.

5. The semantic annotation platform of claim 1, wherein supplementary materials including figures, tables, and video recordings are automatically identified and separated from main text while maintaining links to relevant sections.

6. The semantic annotation platform of claim 1, wherein metadata for figures, tables, and supplementary materials is enriched with at least one standard selected from the group consisting of NBO, NWB, and UMLS identifiers to support cross-study analyses and interoperability.

7. The semantic annotation platform of claim 1, wherein the semantic annotation engine applies AI-driven tagging to legacy publications to align metadata with current standards.

8. The semantic annotation platform of claim 1, further comprising a guided interface for users to verify and adjust AI-generated tags.

9. The semantic annotation platform of claim 1, wherein the semantic annotation engine comprises a cross-ontology linking mechanism that ensures metadata interoperability by mapping terms across multiple ontologies.

10. The semantic annotation platform of claim 1, wherein metadata and data annotations include provenance and licensing information to ensure ethical reuse and regulatory compliance.

11. The semantic annotation platform of claim 1, further comprising APIs and export functionality for integration with external databases and tools.

12. A method for automated publication of heterogeneous biomedical data, the method comprising:
receiving, via a user interface, at least one textual manuscript file and at least one biomedical data file selected from the group consisting of DICOM imaging data, VCF genomic variant data, NWB electrophysiological data, and non-textual data;
extracting structured metadata from the at least one biomedical data file using a semantic annotation engine that applies at least one standardized clinical or biomedical ontology;
incorporating the structured metadata into a semantically enriched article layout that remains machine-readable; and
producing an interactive publication output that simultaneously renders textual narrative and references to the structured biomedical data.

13. The method of claim 12, wherein the method further comprises extracting method elements from a Methods section of the textual manuscript file, resolving each method element to at least one resource object comprising a canonical identifier and a commercial identifier, and generating a bill of materials or bill of services listing resources required to reproduce a study.

14. The method of claim 13, wherein the method further comprises linking each resource object to at least one procurement endpoint selected from the group consisting of purchase, quote request, license acquisition, institutional requisition, data access request, and service scheduling.

15. The method of claim 14, wherein the method further comprises generating a referral identifier for each procurement endpoint and recording transaction metadata when a procurement action is initiated from the publication.

16. The method of claim 13, wherein resolving each method element comprises applying natural language processing to identify resource mentions and tagging each resource with ontology identifiers and commercial identifiers.

17. The method of claim 12, wherein the interactive publication output is compliant with the NIH Data Management and Sharing Policy.

18. A semantic annotation platform for research publications with integrated procurement functionality, the platform comprising:
a semantic annotation engine configured to extract structured metadata from a manuscript file using at least one standardized biomedical ontology;
a method element extraction module configured to parse a Methods section of the manuscript into discrete method steps and resource elements;
a resource resolution module configured to resolve each resource element to a resource object using the semantic annotation engine, the resource object comprising a canonical identifier from the standardized biomedical ontology and a commercial identifier;
a bill of materials generator configured to generate a bill of materials and a bill of services listing resources required to reproduce a study described in the manuscript;
a procurement interface module configured to link each resource object to at least one procurement endpoint; and
a transaction tracking module configured to record transaction metadata when a procurement action is initiated from the publication.

19. The platform of claim 18, wherein the procurement endpoint is selected from the group consisting of purchase, quote request, license acquisition, institutional requisition, data access request, and service scheduling.

20. The platform of claim 18, wherein the transaction tracking module is further configured to generate referral identifiers embedded within the publication and attribute downstream procurement actions back to the publication, author, institution, or platform.
